(19) United States
(12) Patent Application Publication
(10) Pub. No.: US 2019/0135727 A1
(43) Pub. Date: May 9, 2019

(54) Continuous method for producing muconic acid from aldaric acid

(71) Applicant: Teknologian tutkimuskeskus VTT Oy, Espoo (FI)

(72) Inventors: David Thomas, Espoo (FI); Juha Linnekoski, Espoo (FI); Martta Asikainen, Espoo (FI); Olli Jauhiainen, Espoo (FI); Miika Martiskainen, Espoo (FI)

(21) Appl. No.: 16/304,317

(22) PCT Filed: May 31, 2017
(86) PCT No.: PCT/FI2017/050406

§ 371 Date: Nov. 26, 2018

(30) Foreign Application Priority Data
May 31, 2016 (FI) 20165451

(51) Int. Cl. C07C 51/377; C07C 57/16

(52) U.S. Cl. CPC C07C 51/377; C07C 57/16

(57) ABSTRACT

According to an example aspect of the present invention, there is provided a continuous method of producing muconic acid from aldaric acid in the presence of a solid heterogeneous catalyst and an alcohol solvent with short reaction time. Another aspect of the present invention is the use of a non-expensive solid heterogenous catalyst, which is automatically separated from the product.

### FIELD

The present invention relates to a continuous method for producing muconic acid from aldaric acids. Muconic acids are important intermediates in the production of wide variety of industrially significant chemicals and building blocks for such.

### BACKGROUND

The plastic industry traditionally relies on petrochemical platform chemicals for most of its existence. The main problems with petrochemicals are that they are their finite and have detrimental effects upon the environment. Due to increasing environmental regulations, the fluctuating oil price and increasing consumer demand for bio-based chemicals, plastic manufacturers have become increasingly interested in bio-based plastics. Furthermore, oil-based petrochemicals seldom have the functionality (such as acid, aldehyde or ketone groups) needed in many applications, whereas biomass-based components have it naturally. Thus, separate complex oxidation is not needed for bio-based components. Such bio-plastics are plastics that are derived from renewable feedstock such as starch, cellulose, fatty acids, sugars, proteins, and other biological sources. They can be converted into monomers and polymers by microorganisms or chemical reactions. These monomers and polymers are often termed platforms chemicals in that these molecules are used as building blocks to produce many valuable chemicals.

There has lately been a growing interest in muconic acid, which is dicarboxylic acid, due to its potentiality to be used as a platform chemical for many bio-plastics. These include polyurethane and polyethylene terephthalate (Transparency Market Research, 2014). Research has revealed that muconic acid could also be used to produce for example caprolactam, meaning that muconic acid could provide a possible oil free route for e.g. production of nylon (Bui et al., 2012).

Muconic acid has three isometric forms, the trans,trans-muconic acid, cis,trans-muconic acid, and cis,cis-muconic acid as illustrated in scheme 1 below. The reaction to produce caprolactam from muconic acid can be conducted in different ways depending on the isometric form. These isomeric forms can be converted in a two-step route as shown below. Muconic acid is converted in adipic acid with hydrogen and catalyst which is then catalytically reduced to caprolactam with hydrogen and ammonia in the presence of catalyst. Reaction is high yielding, has fewer by-products and avoids sulphate formation produced from crude oil derivatives (Bui et al., 2012 and Transparency Market Research, 2014).

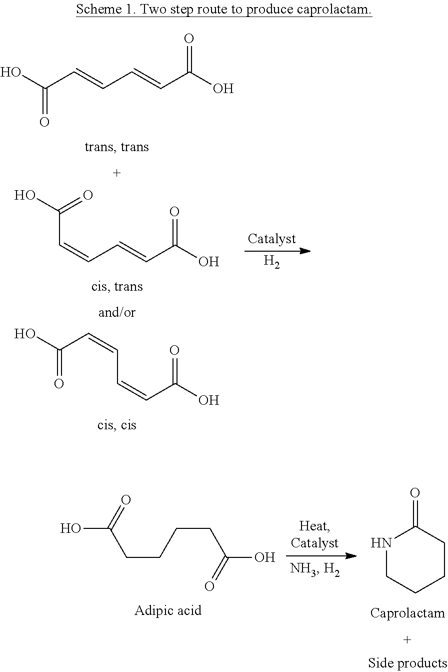 [SMILES: O=C(O)/C=C/C=C/C(=O)O ; O=C(O)/C=C\C=C\C(=O)O ; "H2" ; O=C(O)/C=C\C=C/C(=O)O ; O=C(O)CCCCC(=O)O ; O=C1CCCCN1]

Muconic acid can be prepared chemically and microbiologically. In the chemical route, muconic acid is produced from either sugar or petro-chemical feedstock in the presence of metal catalysts (Pandell, 1976 and Xie et al., 2014). Microbiologically muconic acid can also be produced from aromatic compounds such as toluene, benzene and phenol. Some bacteria are able to convert these chemicals into catechol. Catechol 1,2-dioxygenase enzyme, for example, is able to catalyse cleavage of the aromatic ring to produce muconic acid (U.S. Pat. No. 4,355,107 and Xie et al., 2014). The problem with these processes is the crude oil feedstock, meaning that they cannot be applied for example to the production of bio-nylon.

One route to make muconic from renewable feedstock via is by fermentation of d-glucose. The problem with this microbiological process is its low muconic acid yield. With the existing technology, the achievable bio based muconic acid yield is only 30% (Xie et al., 2014 and Transparency Market Research, 2014). Due to this problem of low yield, muconic acid is seen as a less attractive intermediate in caprolactam production than cyclohexanone. New technology is thus needed to make bio muconic acid route as efficient as petrochemical routes.

WO 2015/189481 presents such new technology and relates to selective catalytic dehydroxylation method of aldaric acids for producing muconic acid and furan chemicals. This method is however limited to batch reaction conditions with long reaction times and limited production capability, and utilizes rather expensive catalysts.

Thus there is a need for a continuous production process, which is able to improve the yields of muconic acid and furthermore make the process more economically feasible.

### SUMMARY OF THE INVENTION

The invention is defined by the features of the independent claims. Some specific embodiments are defined in the dependent claims.

According to a first aspect of the present invention, there is provided a continuous method for producing muconic acid from an aldaric acid raw material.

According to a second aspect of the present invention, there is provided an easily up-scalable process for producing building blocks and platform chemicals from aldaric acids for use for example in the green production of adipic acid, terephthalic acid, hexamethylenediamine, caprolactone, caprolactam, polyamides and nylons.

These and other aspects, together with the advantages thereof over known solutions are achieved by the present invention, as hereinafter described and claimed.

The method according to an embodiment of the present invention is mainly characterized by what is stated in the characterizing part of claim 1.

One advantage of the present invention is that the reaction can be carried out continuously with significantly reduced reaction times, for example from 48 hours (conventional batch reactions) to fewer than 6 hours. Another advantage is the use of a novel solid heterogenous catalyst, which is cheaper compared to the catalysts, such as easily dissolvable trimethyloxorhenium, used in typical batch processes. Further advantage is that the process set up of the present invention enables automatic separation of the catalyst from the product.

Next, the present technology will be described more closely with reference to certain embodiments.

### EMBODIMENTS

The present technology describes a continuous method of producing muconic acid from aldaric acid raw material with the presence of a solvent and solid heterogenous catalyst in a pressurized reactor.

In the present context, the term “heterogeneous catalyst” comprises catalysts having a different phase from that of the reactants. “Phase” herein refers not only to solid, liquid and gas but also to e.g. immiscible liquids. “Weight hourly space velocity” (WHSV) is typically defined as the weight of feed flowing per unit weight of the catalyst per hour.

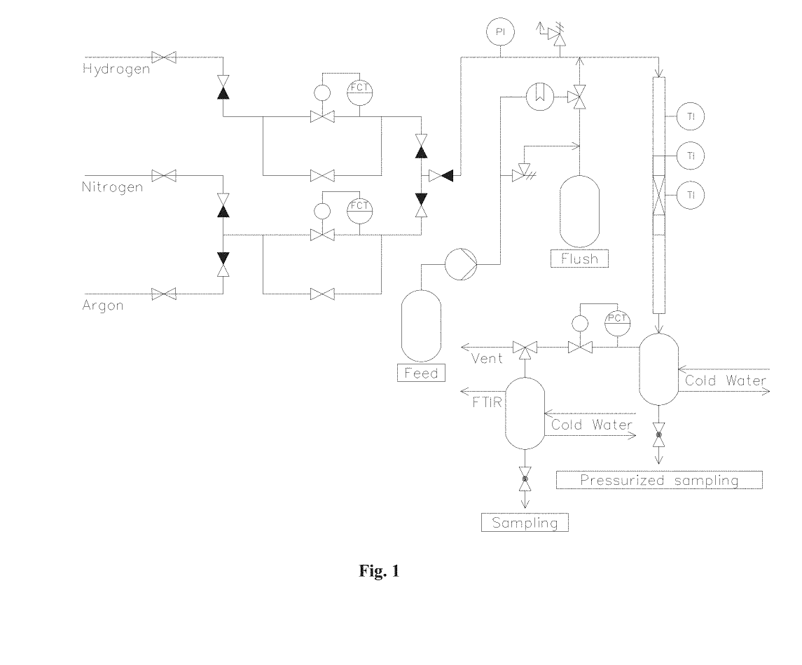

FIG. 1 is a process diagram illustrating one possible process set up of the present invention.

Aldaric acids are a group of sugar acids, where the terminal hydroxyl and aldehyde groups of the sugars have been replaced by terminal carboxylic acids, and are characterized by the formula HOOC—(CHOH)ₙ—COOH, n being an integer from 1 to 10, in particular 1 to 4, such as 3 or 4. The nomenclature of the aldaric acids is based on the sugars from which they are derived. For example, glucose is oxidized to glucaric acid, galactose to galactaric acid and xylose to xylaric acid. Unlike their parent sugars, aldaric acids have the same functional group at both ends of their carbon chain.

According to one embodiment of the present invention a continuous method of producing muconic acid from an aldaric acid comprises passing the aldaric acid in a solvent through a pressurized reactor at temperature of 120 to 140° C. with a solid heterogenous rhenium-based catalyst at weight hourly space velocity of 0.1-10 h⁻¹, or with an aldaric acid feed of 7 to 60 g/h during a pre-determined reaction time.

In another suitable embodiment of the present invention the aldaric acid feed is adjusted to 15 to 30 g/h, more preferably to about 15 g/h (i.e. weight hourly space velocity of 0.2-5 h⁻¹, more preferably to about 0.2 h⁻¹).

One suitable raw material or feedstock for muconic acid production according to one embodiment is galactaric acid having formula I:

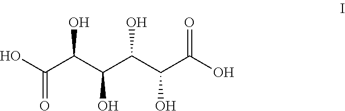 [SMILES: O=C(O)[C@@H](O)[C@H](O)[C@H](O)[C@@H](O)C(=O)O]

Another suitable raw material or feedstock for muconic acid production according to another embodiment is glucaric acid ester having formula II:

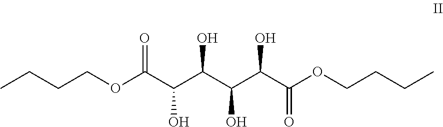 [SMILES: CCCCOC(=O)[C@@H](O)[C@@H](O)[C@H](O)[C@@H](O)C(=O)OCCCC]

Thus, according to one embodiment of the present invention, the aldaric acid used as raw material or feedstock is either galactaric acid or glucaric acid in either free acid or ester form, such as for example glucaric acid butyl ester.

One important aspect of the present invention is the use of a heterogeneous catalyst, which has been found to produce little waste, is easy to separate from the reaction mixture and is also recyclable. Heterogeneous catalysts also permit continuous processing and enable short reaction times in the method herein described.

According to an embodiment of the present invention, one suitable catalyst is ammonium perrhenate, which is preferably fixed in a packed bed inside of the reactor. Such catalyst bed may for example consist of ammonium perrhenate and coarse silicon carbide between quartz wool layers. The catalyst bed is placed into the reactor, which can then be attached into the process system. It is preferred that the catalyst is activated by heat at temperature of 120 to 130° C. for at least an hour before passing the aldaric acid feed through the reactor.

According to another embodiment, the solvent is an alcohol solvent, selected from monovalent or polyvalent C₁-C₆ alcohols, or any combination thereof. Examples of suitable alcohols are ethanol, methanol, 1-butanol or 1-pentanol, or any combination thereof, preferably methanol or butanol. However, also water, dimethyl sulfoxide (DMSO) or tetrahydrofuran (THF) may be used as a solvent.

Thus, according to one preferred embodiment of the present invention the solvent is methanol or butanol.

According to one embodiment, the reactor is pressurized into 500-2000 kPa for example with hydrogen gas, or an inert gas such as argon or nitrogen flow through the reactor. De-pressurization of the reactor may be carried out for example by nitrogen gas.

According to one embodiment, the reaction conditions as described hereinbefore set the reaction time of the present method to 1 to 6 hours.

The purification (i.e. recovering) of the produced products comprises filtering any solid precipitate, washing the precipitate with alcohol and drying the washed product(s) for example by evaporation. The organic phase having the desired product(s) of the present invention is subsequently evaporated and then purified, for example, by silica column chromatography. The results are confirmed by further analysis methods generally known in the art.

According to a further embodiment, the solvent may be regenerated after the reaction from a reaction mixture and is thus reusable. The solvent can be, for example, distilled and reused after the reaction.

According to even further embodiment, the obtained reaction mixture may be recycled back to the reactor and pumped through the catalyst bed again with similar reaction conditions as earlier described, in order to furthermore increase the product yield.

It is to be understood that the embodiments of the invention disclosed are not limited to the particular structures, process steps, or materials disclosed herein, but are extended to equivalents thereof as would be recognized by those ordinarily skilled in the relevant arts. It should also be understood that terminology employed herein is used for the purpose of describing particular embodiments only and is not intended to be limiting.

Reference throughout this specification to one embodiment or an embodiment means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment. Where reference is made to a numerical value using a term such as, for example, about or substantially, the exact numerical value is also disclosed.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various embodiments and example of the present invention may be referred to herein along with alternatives for the various components thereof. It is understood that such embodiments, examples, and alternatives are not to be construed as de facto equivalents of one another, but are to be considered as separate and autonomous representations of the present invention.

Furthermore, the described features, structures, or characteristics may be combined in any suitable manner in one or more embodiments. In the following description, numerous specific details are provided to provide a thorough understanding of embodiments of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

The verbs “to comprise” and “to include” are used in this document as open limitations that neither exclude nor require the existence of also un-recited features. The features recited in depending claims are mutually freely combinable unless otherwise explicitly stated. Furthermore, it is to be understood that the use of “a” or “an”, that is, a singular form, throughout this document does not exclude a plurality.

### INDUSTRIAL APPLICABILITY

At least some embodiments of the present invention find industrial application in generating a full value chain from the forest industry, agriculture, or food industry side streams to platform chemicals and end applications. In principle, this chain comprises production of aldaric acids from aldoses and side-stream carbohydrates, converting the aldaric acids to dicarboxylic acids, which in turn are used as platform chemicals for various bio-based applications, such as bio-based polyesters and nylon, as well as for pharmaceutical building blocks.

### EXAMPLES

The reactor used to study the continuous process is sulphuric free tube reactor. The reactor is 30 cm long and has a diameter of 12 mm. The catalyst bed held with-in the reactor, is supported by a metal rod.

### Experiment Set Up

Raw material in solution is drawn into the system with HPLC pump with the mass flow rate monitored using a balance under the raw material vessel. Before the reactor, raw material flows through pre-heater vaporizer and is mixed with the gas flow. The system is capable of using hydrogen, nitrogen and argon gases. Volumetric flow of the gases is controlled with flow controllers. The reactor heating is done using two 230 V ceramic electronic ovens. The reactor temperature is measured with thermocouple which measures temperature from three points in the reactor. After the reactor, the products enter the pressurized sampling vessel, where the products can be collected under pressure. The products then enter the pressure controller and after that the sampling vessel. In this work, the products were collected using the second sampling vessel. The both sampling vessels are cooled down using a cryostat. The used gas continues from the sampling vessel and to FTIR or an air conditioner. The reactor system diagram is illustrated in FIG. 1.

### Chemicals

All the chemicals were supplied by Sigma-Aldrich, except galactaric acid butyl ester, coarse silicon carbide and quartz wool. Silicon carbide was supplied by Alfa Aesar, quartz wool by Roth and galactaric acid butyl ester was produced in house by the VTT. During the project, more the raw material, galactaric acid butyl ester, had to be synthesised with esterification of D-Saccharic acid potassium salt and n-butanol in the presence of H₂SO₄. The product was oily galactaric acid butyl ester and solid material which had to be filtered with porosity 3 glass filter. GC-FID and GC-MS analysis showed the solid material to be unreacted D-Saccharic acid potassium salt.

### Continuous Experiments Method

The experimental conditions were decided using similar conditions to batch reactor experiments. Due to mucic acid being highly insoluble, a 0.1 g/ml galactaric acid butyl ester in n-butanol solution was used instead as a raw material. The catalyst was changed from MTO to ammonium perrhenate, due to significantly lower catalyst cost and more material required for the continuous reactor. The catalyst bed also consisted of inert silicon carbide to spread the bed to increase the bed height. Changing the solvent from n-butanol to methanol was studied, since it would decrease the process costs and make the process a truly petrochemical free route for muconic acid production. Initially in methanol test, galactaric acid methyl ester was to be used as a raw material, but it was not soluble enough in methanol, whereby butyl ester from had to be used.

### Method

Catalyst bed, consisting of ammonium perrhenate (0.83 g) and coarse silicon carbide (2.49 g) between quartz wool layers (1 g), was placed into the reactor which was then attached into the process system. The reactor ovens were then left to heat up to 140 to 155° C. and the reactor to about 120-130° C. After the heating was complete, the reactor was pressure tested with argon gas by increasing system pressure into 500 to 1000 kPa. The reactor was then pressurized into 500 kPa with hydrogen and hydrogen flow through the reactor was set to 5 l/h. The experiment was started by setting pump raw material feed to 15 g/h and the heating of pre-heater to 115° C. A sample was collected from the product trap every hour. The reactor was stopped by after 6 hours by closing the pump, the hydrogen feed and heating. The reactor was then depressurised and it was set to have 50 l/h nitrogen flow thought it. The end sample was collected about 15 h later.

The method was described for the test 1:15 g/h raw material flow. The other experiments that were performed are shown on table 1.

TABLE 1
Continuous reactor experiments

| Test # | Aim |
|---|---|
| 1 | Raw material flow rate 15 g/h |
| 2 | Raw material flow rate 30 g/h. Sampling was done every |
| | 30 min. |
| 3 | Raw material flow rate 60 g/h. Sampling was done every |
| | 15 min. |
| 4 | Raw material flow rate 7 g/h |
| 5 | The catalyst changed from ammonium perrhenate to MTO |
| | (0.77 g) and P-TSA (0.53 g). |
| 6 | Solvent changed to methanol. Pressure increased into 1000 kPa, |
| | due to volatility of methanol. Reactor heating was decreased |
| | into 135° C. |
| 7 | Recycling system. The products were collected to raw material |
| | vessel and was left to run for 24 h. |
| 8 | Catalyst loading increased: ammonium perrhenate (8.3 g) and |
| | coarse silicon carbide (20.75 g) |

### Analysis

Sample of the reaction material (0.4 ml) was syringed into a glass vial. Pyridine (0.4 ml) was added into the vial and then BSTFA (0.2 ml). The vial was then heated in block heater to 60° C. for 30 min.

GC-FID analyses were carried out using an Agilent 6890 equipped with a FID: Column & length: HP-5 5% Phenyl Methyl Siloxane, 30 m, 0.32 mm, 0.25 um film, carrier gas: He, injector temperature: 250° C., FID temperature: 300° C., oven temperatures: Initial temp: 30° C., Initial time: 1.00 min, Ramp: 13° C./min to 300° C., final time 15 min. GC results were compared to reference standards, which were used to accurately determine the products obtained in the experiments.

### Results

Test 1: Raw material flow rate 15 g/h
GC-FID results can be seen in table 2.

TABLE 2
GC-FID results of continuous reactor test 1

| | Component concentrations (g/l) | | | |
|---|---|---|---|---|
| Test 1 | Galactaric acid | | | Muconic acid |
| Time/h | butyl ester | Mucic acid | Muconic acid | butyl ester |
| 0 | 5.4 | 2.1 | 1.4 | 5.6 |
| 1 | 5.6 | 2.5 | 8.9 | 5.2 |
| 2 | 4.3 | 2.4 | 12.3 | 7.0 |
| 3 | 4.2 | 2.3 | 11.4 | 7.0 |
| 4 | 4.2 | 2.3 | 10.6 | 6.7 |
| 5 | 4.0 | 2.3 | 10.5 | 7.0 |

Test 2: Raw material flow rate 30 g/h
GC-FID results can be seen in table 3.

TABLE 3
GC-FID results of continuous reactor test 2

| | Component concentrations (g/l) | | | |
|---|---|---|---|---|
| Test 2 | Galactaric acid | | | Muconic acid |
| Time/h | butyl ester | Mucic acid | Muconic acid | butyl ester |
| 0 | 5.4 | 2.1 | 1.4 | 5.6 |
| 1 | 2.2 | 2.4 | 9.2 | 6.4 |
| 2 | 1.6 | 2.2 | 7.4 | 6.5 |
| 3 | 2.2 | 2.2 | 7.8 | 6.4 |
| 4 | 2.2 | 2.2 | 8.3 | 6.6 |
| 5 | 2.4 | 2.3 | 8.8 | 6.8 |
| 6 | 3.4 | 2.4 | 8.7 | 6.7 |

Test 3: Raw material flow rate 60 g/h
GC-FID results can be seen in table 4.

TABLE 4
GC-FID results of continuous reactor test 3

| | Component concentrations (g/l) | | | |
|---|---|---|---|---|
| Test 3 | Galactaric acid | | | Muconic acid |
| Time/h | butyl ester | Mucic acid | Muconic acid | butyl ester |
| 0 | 5.4 | 2.1 | 1.4 | 5.6 |
| 1 | 3.5 | 2.7 | 13.0 | 7.5 |
| 2 | 2.1 | 2.4 | 10.2 | 7.5 |
| 3 | 2.2 | 2.3 | 7.9 | 7.0 |
| 4 | 2.8 | 2.4 | 8.4 | 7.1 |
| 5 | 2.3 | 2.4 | 8.7 | 7.3 |
| 6 | 2.7 | 2.4 | 8.8 | 7.0 |

Test 4: Raw material flow rate 7 g/h
GC-FID results can be seen in table 5.

TABLE 5
GC-FID results of continuous reactor test 4

| | Component concentrations (g/l) | | | |
|---|---|---|---|---|
| Test 4 | Galactaric acid | | | Muconic acid |
| Time/h | butyl ester | Mucic acid | Muconic acid | butyl ester |
| 0 | 5.4 | 2.1 | 1.4 | 5.6 |
| 1 | 3.2 | 1.2 | 2.9 | 2.4 |
| 2 | 7.1 | 2.5 | 15.3 | 7.2 |
| 3 | 8.3 | 2.7 | 15.2 | 7.8 |
| 4 | 8.5 | 2.5 | 15.3 | 7.4 |
| 5 | 8.1 | 2.6 | 12.8 | 7.1 |

Test 5: Catalyst changed to MTO
GC-FID results can be seen in table 6.

TABLE 6
GC-FID results of continuous reactor test 5

| | Component concentrations (g/l) | | | |
|---|---|---|---|---|
| Test 5 | Galactaric acid | | | Muconic acid |
| Time/h | butyl ester | Mucic acid | Muconic acid | butyl ester |
| 0 | 5.4 | 2.1 | 1.4 | 5.6 |
| 1 | 2.5 | 1.6 | 23.3 | 18.5 |
| 2 | 1.9 | 2.2 | 21.8 | 9.1 |
| 3 | 1.7 | 2.1 | 13.1 | 6.7 |
| 4 | 2.1 | 2.1 | 7.7 | 6.5 |
| 5 | 3.3 | 2.3 | 6.4 | 6.8 |
| 6 | 4.4 | 2.2 | 4.9 | 6.3 |

Test 6: Solvent changed to methanol
GC-FID results can be seen in table 7.

TABLE 7
GC-FID results of continuous reactor test 6

| | Component concentrations (g/l) | | | |
|---|---|---|---|---|
| Test 6 | Galactaric acid | | | Muconic acid |
| Time/h | butyl ester | Mucic acid | Muconic acid | methyl ester |
| 1 | 0.0 | 0.4 | 6.8 | 0.5 |
| 2 | 1.9 | 2.2 | 19.8 | 1.9 |
| 3 | 2.8 | 2.7 | 19.0 | 1.9 |
| 4 | 5.5 | 3.9 | 19.0 | 1.9 |

Test 7: Recycling reactor
GC-FID results can be seen in table 8.

TABLE 8
GC-FID results of continuous reactor test 7

| | Component concentrations (g/l) | | | |
|---|---|---|---|---|
| Test 7 | Galactaric acid | | | Muconic acid |
| Time/h | butyl ester | Mucic acid | Muconic acid | butyl ester |
| 2.5 | 8.7 | 3.7 | 13.4 | 4.6 |
| 18 | 9.2 | 4.1 | 18.8 | 5.7 |
| 26 | 9.6 | 4.2 | 21.0 | 6.7 |

Test 8: Catalyst amount increase
GC-FID results can be seen in table 9.

TABLE 9
GC-FID results of continuous reactor test 8

| | Component concentrations (g/l) | | | |
|---|---|---|---|---|
| Test 8 | Galactaric acid butyl | Mucic | Muconic | Muconic acid butyl |
| Time/h | ester | acid | acid | ester |
| 1 | 2.9 | 0.1 | 2.6 | 1.2 |
| 2 | 15.0 | 3.7 | 8.2 | 3.0 |
| 3 | 16.6 | 4.2 | 8.6 | 3.3 |
| 4 | 17.1 | 4.2 | 8.3 | 3.1 |

### CITATION LIST

### Patent Literature

U.S. Pat. No. 4,355,107
WO 2015/189481

### Non Patent Literature

1. Bui, Vu; COUDRAY, Laetitia; Frost, John W, inventors; Amyris, Inc., assignee. Process for preparing caprolactam and polyamides therefrom. World patent WO 20,12,141,997. 2012 October 18.
2. Transparency Market Research. Muconic Acid Market: Global Industry Analysis, Size, Share, Growth, Trends and Forecast 2014-2020. Albany (US): Transparency Market Research; 2014 Oct. 8. 56 p.
3. Pandell, Alexander J. Enzymic-like aromatic oxidations. Metal-catalyzed peracetic acid oxidation of phenol and catechol to cis,cis-muconic acid. The Journal of Organic Chemistry. 1976 December; 41(25).
4. Xie, Neng-Zhong; Liang, Hong; Huang, Ri-Bo; Xu, Ping. Biotechnological production of muconic acid: current status and future prospects. Biotechnology Advances. 2014 May-June; 32(3).

1. A continuous method of producing muconic acid from an aldaric acid comprising passing the aldaric acid in a solvent through a pressurized reactor at temperature of 120 to 140° C. with a solid heterogenous rhenium-based catalyst at weight hourly space velocity of 0.1-10 h⁻¹ during a pre-determined reaction time.

2. The method according to claim 1, wherein the aldaric acid is galactaric acid or glucaric acid, either in free acid or ester form.

3. The method according to claim 1, wherein the solvent is an alcohol solvent selected from monovalent or polyvalent C₁-C₆ alcohols, or any combination thereof.

4. The method according to claim 1, wherein the solvent is methanol or butanol.

5. The method according to claim 1, wherein the catalyst is ammonium perrhenate, which is fixed in a packed bed inside of the reactor.

6. The method according to claim 1, wherein the reactor is pressurized into 500-2000 kPa with hydrogen, argon or nitrogen gas flow through the reactor.

7. The method according to claim 1, wherein the catalyst is activated by heat at temperature of 120 to 130° C. for at least an hour before passing the aldaric acid feed through the reactor.

8. The method according to claim 1, wherein the weight hourly space velocity is 0.2-5 h⁻¹, more preferably about 0.2 h⁻¹.

9. The method according to claim 1, wherein the reaction time is 1 to 6 hours.

10. Use of muconic acid and esters as an intermediate in the production of industrial chemicals and pharmaceutical building blocks wherein the muconic acid and esters are produced by a continuous method of producing muconic acid from an aldaric acid comprising passing the aldaric acid in a solvent through a pressurized reactor at temperature of 120 to 140° C. with a solid heterogenous rhenium-based catalyst at weight hourly space velocity of 0.1-10 h⁻¹ during a pre-determined reaction time.

11. Use of muconic acid and esters as an intermediate in the production of chemicals selected form the group consisting of: adipic acid, terephthalic acid, hexamethylenediamine, caprolactone, caprolactam, polyamides and nylons, wherein the muconic acid and esters are produced by a continuous method of producing muconic acid from an aldaric acid comprising passing the aldaric acid in a solvent through a pressurized reactor at temperature of 120 to 140° C. with a solid heterogenous rhenium-based catalyst at weight hourly space velocity of 0.1-10 h⁻¹ during a pre-determined reaction time.
